(19) United States
(12) Patent Application Publication
(10) Pub. No.: US 2021/0277405 A1
(43) Pub. Date: Sep. 9, 2021

(54) CONTROLLABLE PROTEIN DEGRADATION VIA ENGINEERING DEGRADATION TAG VARIANTS IN CORYNEBACTERIUM HOST CELLS

(71) Applicants: Conagen Inc., Bedford, MA (US); Sumitomo Chemical Co., Ltd., Tokyo (JP)

(72) Inventors: Ilya Tikh, Stoneham, MA (US); Sonya Clarkson, Beverly, MA (US); Brendon Dusel, West Roxbury, MA (US); Jacob Edward Vick, Cambridge, MA (US); Xiaodan Yu, Lexington, MA (US); Bryan Salas-Santiago, Lowell, MA (US)

(73) Assignees: Conagen Inc., Bedford, MA (US); Sumitomo Chemical Co., Ltd., Tokyo (JP)

(21) Appl. No.: 17/205,008

(22) Filed: Mar. 18, 2021

Related U.S. Application Data
(63) Continuation of application No. PCT/US2019/052032, filed on Sep. 19, 2019.
(60) Provisional application No. 62/733,521, filed on Sep. 19, 2018.

(51) Int. Cl. C12N 15/77; C12P 1/04

(52) U.S. Cl. CPC C12N 15/77; C12N 2800/101; C12P 1/04

(57) ABSTRACT

The present invention relates to the creation of a control system within a host cell to limit or eliminate degradation of key specified products at certain times during fermentation and to redirect the metabolic flux of the cell toward higher production of same key specified products.

### CROSS-REFERENCE TO RELATED APPLICATIONS

This application claims priority to U.S. Provisional Patent Application No. 62/733,521, filed on Sep. 19, 2018, the content of which is incorporated by reference herein in its entirety.

### FIELD OF THE INVENTION

The present invention relates to the areas of microbial genetics and recombinant DNA technology. The present teachings provide polynucleotide sequences, polypeptide sequences, vectors, microorganisms, and methods useful for inducing and regulating protein degradation controllably in bacterial cells, specifically, in Corynebacterium spp.

### BACKGROUND OF THE INVENTION

In an unmodified cell, the amount of proteins present at different points in the cellular life cycle are a function not only of protein synthesis, but also of protein degradation. With this in mind, it is not surprising that the half-lives of proteins within cells vary widely, from minutes to several days, and differential rates of protein degradation are an important aspect of the cellular regulatory apparatus. For example, regulatory molecules, such as transcription factors, are rapidly degraded to allow the cell to respond quickly to changing conditions in its environment. Other proteins are rapidly degraded in response to specific metabolic signals, providing another mechanism for the regulation of intracellular enzyme activity. In addition, faulty or damaged proteins are recognized and rapidly degraded within cells, thereby eliminating or limiting the consequences of mistakes made during protein synthesis.

In bacterial systems, protein degradation occurs to remove damaged and/or misfolded proteins. A system that functions in this capacity is the ssrA-mediated tagging degradation system. The ssrA tag, an 11-aa peptide added to the C-terminus of proteins stalled during translation, targets proteins for degradation by the proteases ClpXP and ClpAp. The ssrA tag interacts with SspB, a specificity-enhancing factor (also known as an adaptor protein) for ClpX. SspB and ClpX work together to recognize ssrA-tagged substrates for proteolysis.

However, native protein degradation system often works too efficiently, as proteolytic degradation can be triggered by the ssrA-mediated tagging alone. The art therefore seeks improved methods where protein degradation can be better controlled, including better control over the degradation rate of specific substrates and the timing of the degradation in specific metabolic phases.

### SUMMARY OF THE INVENTION

The present invention encompasses improved methods of increasing the titer and/or yield of a desired product produced by an engineered microbial organism. Such enhancement is achieved by inducing the degradation of a target enzyme, where the target enzyme either metabolizes the desired product or the target enzyme functions as a negative feedback for the synthetic pathway used to produce the desired product. Because the target enzyme can be an essential enzyme during the growth phase of the microbial organism, it is critical that the degradation of the target enzyme does not occur significantly until cell growth is stabilized. Once growth of the microbial organism can be slowed or stopped, the degradation of the target enzyme can then be induced. The present invention achieves this objective by recombinantly engineering the microbial organism to express a heterologous protein degradation system that includes an adaptor protein and a degradation tag, where the expression of the adaptor protein can be induced at a desired time point to trigger proteolysis.

Accordingly, in one aspect, the present invention provides a microbial organism that has been recombinantly engineered to express a heterologous protein degradation system that includes an adaptor protein and a degradation tag. In some embodiments, the microbial organism is a Corynebacterium species host cell. In certain embodiments, the microbial organism is Corynebacterium glutamicum. In some embodiments, the heterologous protein degradation system includes an adaptor protein obtained from Staphylococcus aureus or a functional variant thereof. For example, the adaptor protein can be a TrfA adaptor protein comprising an amino acid sequence having at least 80%, 85%, 90%, 92%, 95%, 96%, 97%, 98%, or 99% sequence identity to SEQ ID NO. 4. In some embodiments, the heterologous protein degradation system includes a degradation tag including, in a 5′ to 3′ direction, an adaptor binding region, an optional spacer region, and a protease recognition region, wherein the adaptor binding region specifically binds the TrfA adaptor protein. The protease recognition region of the degradation tag allows a target protein tagged by the degradation tag to be recognized by a protease such as ClpCP, ClpXP, or ClpAP. In preferred embodiments, the protease is a protease native in the host cell. Notably, significant degradation does not occur until the expression of the TrfA adaptor protein is induced, and the trfA adaptor protein binds to the adaptor binding region of the degradation tag. In other words, the heterologous protein degradation system of the present invention ensures that signification degradation of the target protein only takes place when (1) the target protein is tagged by a degradation tag according to the present invention and (2) the expression of a corresponding adaptor protein is induced. For example, significant degradation can be measured by observing that the amount of the target protein is reduced by at least 15%, 20%, 30%, 40%, 50%, 60%, 70%, 80%, or 90% after the adaptor protein is induced, compared to before expression of the adaptor protein.

In various embodiments, the degradation tag according to the present teachings is a variant of an S. aureus degradation tag having the amino acid sequence of SEQ ID NO. 22. More specifically, the present degradation tag variant includes, as the last three amino acids of its C-terminus, an amino acid sequence selected from the group consisting of DQP, KPS, DGA, DGS, DQA, KNP, QPS, MKP, DQS, and HPP. For example, the present degradation tag variant can include the amino acid sequence of SEQ ID NO. 24, SEQ ID NO. 26, SEQ ID NO. 28, SEQ ID NO. 30, SEQ ID NO. 32, SEQ ID NO. 34, SEQ ID NO. 47, SEQ ID NO. 48, SEQ ID NO. 49, SEQ ID NO. 50, SEQ ID NO. 51, SEQ ID NO. 52, SEQ ID NO. 53, SEQ ID NO. 54, SEQ ID NO. 55, SEQ ID NO. 56, SEQ ID NO. 57, or SEQ ID NO. 58. In certain embodiments, the present degradation tag variant can include the amino acid sequence of SEQ ID NO. 30 or SEQ ID NO. 32. In certain embodiments, the present degradation tag variant can include the amino acid sequence of SEQ ID NO. 28, SEQ ID NO. 34, SEQ ID NO. 47, SEQ ID NO. 48, SEQ ID NO. 49, SEQ ID NO. 50, SEQ ID NO. 51, SEQ ID NO. 52, SEQ ID NO. 53, SEQ ID NO. 54, SEQ ID NO. 55, SEQ ID NO. 56, SEQ ID NO. 57, or SEQ ID NO. 58. In certain embodiments, the present degradation tag variant can include the amino acid sequence of SEQ ID NO. 24 or SEQ ID NO. 26.

In some embodiments, the present invention provides a microbial organism that has been recombinantly engineered to express a heterologous protein degradation system that includes an adaptor protein obtained from Escherichia coli or a functional variant thereof. For example, the adaptor protein can be a SspB adaptor protein comprising an amino acid sequence having at least 80%, 85%, 90%, 92%, 95%, 96%, 97%, 98%, or 99% sequence identity to SEQ ID NO. 2. In some embodiments, the heterologous protein degradation system includes a degradation tag including, in a 5′ to 3′ direction, an adaptor binding region, an optional spacer region, and a protease recognition region, wherein the adaptor binding region specifically binds the SspB adaptor protein. The protease recognition region of the degradation tag allows a target protein tagged by the degradation tag to be recognized by a protease such as ClpCP, ClpXP, or ClpAP. In preferred embodiments, the protease is a protease native in the host cell. Notably, significant degradation does not occur until the expression of the SspB adaptor protein is induced, and the SspB adaptor protein binds to the adaptor binding region of the degradation tag. For example, significant degradation can be measured by observing that the amount of the target protein is reduced by at least 15%, 20%, 30%, 40%, 50%, 60%, 70%, 80%, or 90% after the adaptor protein is induced, compared to before expression of the adaptor protein.

In various embodiments, the degradation tag according to the present teachings is a variant of an E. coli degradation tag having the amino acid sequence of SEQ ID NO. 8. More specifically, the present degradation tag variant includes, as the last three amino acids of its C-terminus, an amino acid sequence selected from the group consisting of DQP, KPS, DGA, DGS, DQA, KNP, QPS, MKP, DQS, and HPP. For example, the present degradation tag variant can include the amino acid sequence of SEQ ID NO. 10, SEQ ID NO. 12, SEQ ID NO. 14, SEQ ID NO. 16, SEQ ID NO. 18, SEQ ID NO. 20, SEQ ID NO. 35, SEQ ID NO. 36, SEQ ID NO. 37, SEQ ID NO. 38, SEQ ID NO. 39, SEQ ID NO. 40, SEQ ID NO. 41, SEQ ID NO. 42, SEQ ID NO. 43, SEQ ID NO. 44, SEQ ID NO. 45, or SEQ ID NO. 46. In certain embodiments, the present degradation tag variant can include the amino acid sequence of SEQ ID NO. 16 or SEQ ID NO. 18. In certain embodiments, the present degradation tag variant can include the amino acid sequence of SEQ ID NO. 14, SEQ ID NO. 20, SEQ ID NO. 35, SEQ ID NO. 36, SEQ ID NO. 37, SEQ ID NO. 38, SEQ ID NO. 39, SEQ ID NO. 40, SEQ ID NO. 41, SEQ ID NO. 42, SEQ ID NO. 43, SEQ ID NO. 44, SEQ ID NO. 45, or SEQ ID NO. 46. In certain embodiments, the present degradation tag variant can include the amino acid sequence of SEQ ID NO. 10 or SEQ ID NO. 12.

In some embodiments, the present invention provides a microbial organism that has been recombinantly engineered to express two separate heterologous protein degradation systems, specifically, a first protein degradation system that includes a first adaptor protein and a first degradation tag variant, and a second protein degradation system that includes a second adaptor protein and a second degradation tag variant. The first and second heterologous protein degradation systems can function orthogonally, such that each targets different target proteins and there is minimal cross-talk, e.g., the first adaptor protein does not target a protease recognized by the second degradation tag variant or vice versa.

According to such embodiments, the first adaptor protein can be obtained from Staphylococcus aureus or can be a functional variant thereof. For example, the first adaptor protein can be a TrfA adaptor protein comprising an amino acid sequence having at least 80%, 85%, 90%, 92%, 95%, 96%, 97%, 98%, or 99% sequence identity to SEQ ID NO. 4. In some embodiments, the first degradation tag variant can include, in a 5′ to 3′ direction, an adaptor binding region, an optional spacer region, and a protease recognition region, wherein the adaptor binding region specifically binds the TrfA adaptor protein. The protease recognition region of the degradation tag allows a target protein tagged by the degradation tag to be recognized by a protease such as ClpCP, ClpXP, or ClpAP. The first degradation tag can be a variant of an S. aureus degradation tag having the amino acid sequence of SEQ ID NO. 22. More specifically, the first degradation tag variant can include, as the last three amino acids of its C-terminus, an amino acid sequence selected from the group consisting of DQP, KPS, DGA, DGS, DQA, KNP, QPS, MKP, DQS, and HPP. For example, the first degradation tag variant can include the amino acid sequence of SEQ ID NO. 24, SEQ ID NO. 26, SEQ ID NO. 28, SEQ ID NO. 30, SEQ ID NO. 32, SEQ ID NO. 34, SEQ ID NO. 47, SEQ ID NO. 48, SEQ ID NO. 49, SEQ ID NO. 50, SEQ ID NO. 51, SEQ ID NO. 52, SEQ ID NO. 53, SEQ ID NO. 54, SEQ ID NO. 55, SEQ ID NO. 56, SEQ ID NO. 57, or SEQ ID NO. 58. The second adaptor protein can be obtained from Escherichia coli or can be a functional variant thereof. For example, the second adaptor protein can be an SspB adaptor protein comprising an amino acid sequence having at least 80%, 85%, 90%, 92%, 95%, 96%, 97%, 98%, or 99% sequence identity to SEQ ID NO. 2. In some embodiments, the second degradation tag can include, in a 5′ to 3′ direction, an adaptor binding region, an optional spacer region, and a protease recognition region, wherein the adaptor binding region specifically binds the SspB adaptor protein. The protease recognition region of the degradation tag allows a target protein tagged by the degradation tag to be recognized by a protease such as ClpCP, ClpXP, or ClpAP. The second degradation tag can be a variant of an E. coli degradation tag having the amino acid sequence of SEQ ID NO. 8. More specifically, the second degradation tag variant can include, as the last three amino acids of its C-terminus, an amino acid sequence selected from the group consisting of DQP, KPS, DGA, DGS, DQA, KNP, QPS, MKP, DQS, and HPP. For example, the second degradation tag variant can include the amino acid sequence of SEQ ID NO. 10, SEQ ID NO. 12, SEQ ID NO. 14, SEQ ID NO. 16, SEQ ID NO. 18, SEQ ID NO. 20, SEQ ID NO. 35, SEQ ID NO. 36, SEQ ID NO. 37, SEQ ID NO. 38, SEQ ID NO. 39, SEQ ID NO. 40, SEQ ID NO. 41, SEQ ID NO. 42, SEQ ID NO. 43, SEQ ID NO. 44, SEQ ID NO. 45, or SEQ ID NO. 46.

Another aspect of the present invention provides a method of controlling the degradation of a first target protein in a microbial organism such as a Corynebacterium species host cell, where the host cell has been recombinantly engineered to express a first heterologous protein degradation system that includes a first adaptor protein and a first degradation tag variant, and where the host cell also has been recombinantly engineered to produce a first product via a first heterologous biosynthetic pathway. The method can include (i) expressing the first degradation tag variant adapted to tag the first target protein; (ii) growing the host cell until a desired growth rate is reached; and (iii) inducing the expression of the first adaptor protein, where the first adaptor protein can be a TrfA adaptor protein comprising an amino acid sequence having at least 80%, 85%, 90%, 92%, 95%, 96%, 97%, 98%, or 99% sequence identity to SEQ ID NO. 4. After the expression of the TrfA adaptor protein is induced, the amount of the first target protein present in the host cell can decrease by at least 15%, 20%, 30%, 40%, 50%, 60%, 70%, 80%, or 90%. The decrease can be caused by degradation by a protease selected from the group consisting of ClpCP, ClpXP and ClpAP.

In various embodiments, the first target protein can be an essential protein for the growth of the host cell. In some embodiments, the presence of the first target protein can function as a negative feedback in the first heterologous biosynthetic pathway for producing the first product. In other embodiments, the first target protein can metabolize the first product, thereby reducing the collectible amount of the first product.

In various embodiments, the expression of the TrfA adaptor protein can be induced by a temperature change, a pH change, exposure to light and/or by changing the level of a given molecule within the host cell. In various embodiments, the last three amino acid sequence of the C-terminus of the first degradation tag can be selected from the group consisting of DQP, KPS, DGA, DGS, DQA, KNP, QPS, MKP, DQS, and HPP.

In some embodiments, the present method can involve a host cell that has been further recombinantly engineered to express a second protein degradation system that includes a second adaptor protein and a second degradation tag variant, and the host cell also has been recombinantly engineered to produce a second product via a second heterologous biosynthetic pathway. The method can include (iv) expressing the second degradation tag variant adapted to tag the second target protein; and (v) after the host cell has reached a desired growth rate, inducing the expression of the second adaptor protein, where the second adaptor protein can be a SspB adaptor protein comprising an amino acid sequence having at least 80%, 85%, 90%, 92%, 95%, 96%, 97%, 98%, or 99% sequence identity to SEQ ID NO. 2.

After the expression of the SspB adaptor protein is induced, the amount of the second target protein present in the host cell can decrease by at least 15%, 20%, 30%, 40%, 50%, 60%, 70%, 80%, or 90%. The decrease can be caused by degradation by a protease selected from the group consisting of ClpCP, ClpXP and ClpAP.

In various embodiments, the second target protein can be an essential protein for the growth of the host cell. In some embodiments, the presence of the second target protein can function as a negative feedback in the second heterologous biosynthetic pathway for producing the second product. In other embodiments, the second target protein can metabolize the second product, thereby reducing the collectible amount of the second product.

In various embodiments, the expression of the SspB adaptor protein can be induced by a temperature change, a pH change, exposure to light and/or by changing the level of a given molecule within the host cell. In various embodiments, the last three amino acid sequence of the C-terminus of the second degradation tag can be selected from the group consisting of DQP, KPS, DGA, DGS, DQA, KNP, QPS, MKP, DQS, and HPP.

In various embodiments, the first product and/or the second product can be an amino acid selected from the group consisting of methionine, glutamate, lysine, threonine, isoleucine, arginine, and cysteine. In certain embodiments, the first product and/or the second product can be an L-amino acid selected from the group consisting of L-methionine, L-glutamate, L-lysine, L-threonine, L-isoleucine, L-arginine, and L-cysteine.

While the disclosure is susceptible to various modifications and alternative forms, specific embodiments thereof are shown by way of example in the drawings and will herein be described in detail. It should be understood, however, that the drawings and detailed description presented herein are not intended to limit the disclosure to the particular embodiment disclosed, but on the contrary, the intention is to cover all modifications, equivalents, and alternatives falling within the spirit and scope of the present disclosure as defined by the appended claims.

Other features and advantages of this invention will become apparent in the following detailed description of preferred embodiments of this invention, taken with reference to the accompanying drawings.

### BRIEF DESCRIPTION OF THE DRAWINGS

FIG. 1 illustrates how a degradation tag 20 is operably linked to a target protein 10. The degradation tag usually is located near the C terminus of the target protein. The degradation tag 20 includes an adapter binding region 202, an optional spacer region 204, and a protease recognition region 206.

FIG. 2 demonstrates how the ssrA degradation tag (wild-type E. coli) and its variants (i.e., those with synthetically derived Clp protease recognition sequences) can have different modulating effects on the degradation of a tagged target protein and how the expression of the adapter protein SspB (from wild-type E. coli) can be induced and paired with the use of the ssrA degradation tag and its variants to further allow dynamic control the degradation of the tagged target protein. Specifically, the reporter protein mCherry was used as the target protein and its fluorescence signal was measured to allow quantitative comparisons. Fluorescence measurements obtained with tagged mCherry were normalized to those obtained with wild-type mCherry (first two bars on the left). Each data set includes measurement without induction SspB (left) and with induction of SspB (right). In FIG. 2, the gene expression of mCherry was driven by a strong promoter (specifically, pSOD).

FIG. 3 demonstrates how the ssrA degradation tag (wild-type E. coli) and its variants (i.e., those with synthetically derived Clp protease recognition sequences) can have different modulating effects on the degradation of a tagged target protein and how the expression of the adapter protein SspB (from wild-type E. coli) can be induced and paired with the use of the ssrA degradation tag and its variants to further allow dynamic control the degradation of the tagged target protein. Specifically, the reporter protein mCherry was used as the target protein and its fluorescence signal was measured to allow quantitative comparisons. Fluorescence measurements obtained with tagged mCherry were normalized to those obtained with wild-type mCherry (first two bars on the left). Each data set includes measurement without induction SspB (left) and with induction of SspB (right). In FIG. 3, the gene expression of mCherry was driven by a weak promoter (specifically, Min5).

FIG. 4 demonstrates how the trfA degradation tag (wild-type S. aureus) and its variants (i.e., those with synthetically derived Clp protease recognition sequences) can have different modulating effects on the degradation of a tagged target protein and how the expression of the adapter protein TrfA (from wild-type S. aureus) can be induced and paired with the use of the trfA degradation tag and its variants to further allow dynamic control the degradation of the tagged target protein. Specifically, the reporter protein mCherry was used as the target protein and its fluorescence signal was measured to allow quantitative comparisons. Fluorescence measurements obtained with tagged mCherry were normalized to those obtained with wild-type mCherry (first two bars on the left). Each data set includes measurement without induction SspB (left) and with induction of SspB (right). In FIG. 4, the gene expression of mCherry was driven by a strong promoter (specifically, pSOD).

FIG. 5 demonstrates how the trfA degradation tag (wild-type S. aureus) and its variants (i.e., those with synthetically derived Clp protease recognition sequences) can have different modulating effects on the degradation of a tagged target protein and how the expression of the adapter protein TrfA (from wild-type S. aureus) can be induced and paired with the use of the trfA degradation tag and its variants to further allow dynamic control the degradation of the tagged target protein. Specifically, the reporter protein mCherry was used as the target protein and its fluorescence signal was measured to allow quantitative comparisons. Fluorescence measurements obtained with tagged mCherry were normalized to those obtained with wild-type mCherry (first two bars on the left). Each data set includes measurement without induction SspB (left) and with induction of SspB (right). In FIG. 4, the gene expression of mCherry was driven by a weak promoter (specifically, Min5).

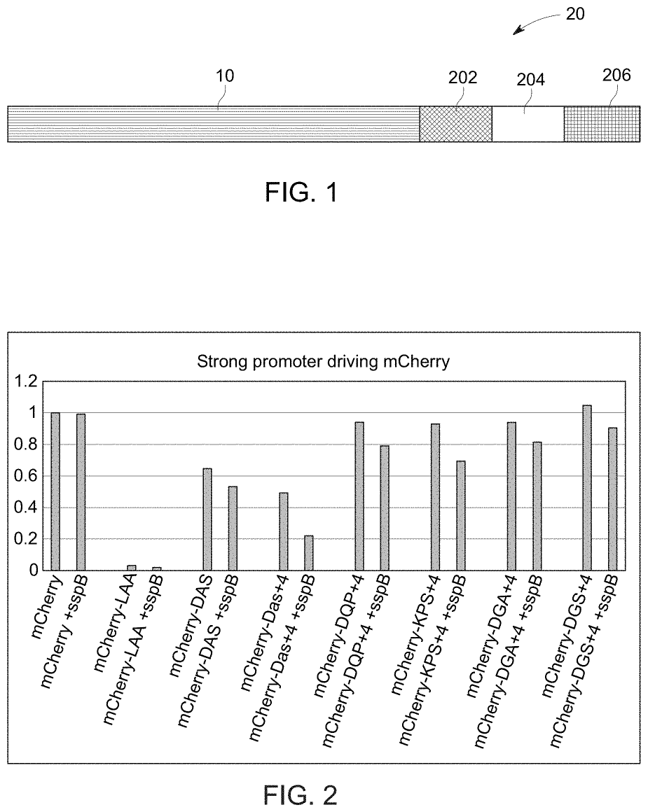

### DETAILED DESCRIPTION

Staphylococcus aureus trfA. S. aureus trfA is an adaptor gene related to the proteolytic adaptor protein mecA of Bacillus subtilis encoding an adaptor protein implicated in multiple roles, notably, proteolysis and genetic competence. Its deletion leads to almost complete loss of resistance to oxacillin and glycopeptide antibiotics in glycopeptide-intermediate S. aureus (GISA) derivatives of methicillin-susceptible or methicillin-resistant (MRSA) clinical or laboratory isolates. Importantly, the TrfA adaptor protein has been found to interact with ClpCP to help control protein degradation in S. aureus.

Specificity-enhancing factor SspB. The SspB adaptor protein is present in a wide range of organisms and directs ssrA-tagged proteins for degradation by cellular proteases, frequently ClpXP or ClpCP protease complexes. The interaction of SspB with ClpXP has been shown to further enhance the activity of the protease complex.

ClpXP. ClpXP is a protein complex formed of four ClpX subunits, which function to recognize and bind unstructured proteins, and six ClpP subunits, which function as an ATP dependent protease. The ClpXP complex is found across both Gram-positive and Gram-negative organisms and is one of the primary quality control mechanisms for protein expression in bacteria.

Previous research has shown effective protein degradation in E. coli by addition of the ssrA degradation tag to the C-terminus of the target protein. Control is achieved via SspB, which as described above, is an adaptor protein required for efficient binding of the ssrA tag to the ClpXP protease complex, the expression of which can be tightly controlled by an exogenous inducer. Additionally, varying the last three amino acids of the ssrA tag has been shown to modulate the efficiency and rate of the targeted protein degradation with and without the SspB adaptor protein.

Nevertheless, to the inventors' knowledge, there has not been specific reports in the literature successfully demonstrating controllable, inducible heterologous protein degradation systems in Corynebacterium glutamicum. Furthermore, the inventors have developed novel degradation tag variants that by themselves do not trigger degradation by native proteases (or only minimally), but upon induced expression of E. coli SspB and/or S. aureus TrfA target tagged substrates for significant degradation by such native proteases.

Corynebacterium glutamicum was isolated in 1957 in Japan due to its ability to excrete large amounts of the amino acid L-glutamate under a biotin limitation. Within the last several decades C. glutamicum also has been modified not only to be an excellent production platform for amino acids but also for a variety of other metabolites, including organic acids. Moreover, Corynebacterium's intrinsic characteristics make it an excellent selection for large scale commercial production. Such intrinsic characteristics include its lack of pathogenicity and its lack of spore-forming ability, both desirable traits as listed by the U.S. Center for Biologics Evaluation and Research and the U.S. Center for Drug Evaluation and Research guidelines, as well as its high growth rate, its relatively limited growth requirements, the absence of autolysis in certain industrial strains under low-growth conditions, the relative stability of the corynebacterial genome itself and the absence of native extracellular protease secretions contribute in making Corynebacteria a very good host for industrial-scale protein expression.

Despite these promising attributes, the development of Corynebacterium as a platform for synthetic biology production has been hampered by the lack of available synthetic biology tools to predictively control gene transcription, protein degradation, translation, and the overall activity of desired pathways without compromising essential cell functions. In addition, attempts to develop and fully characterize the performance of the diverse genetic circuits in Corynebacterium has not yet been completed. Moreover, many of the tools developed and perfected in E. Coli or other organisms do not always directly transfer or correlate to Corynebacterium, requiring significant ‘work arounds’ to develop similar functionality in Corynebacterium as a platform organism. Thus, the development of tools to tune genetic circuits, such as the ssrA tagging system, is necessary to fully unlock the metabolic capacity of Corynebacterium for the production of value-added compounds.

In addition, the tunable control of native metabolic enzyme levels is a critical aspect of engineering Corynebacterium spp strains for the production of heterologous compounds, such as biofuels, biopolymers, and molecules with therapeutic properties. In this situation, knockouts may lead to cell death or failure to produce high titers of the desired compounds while static knockdown may lead to undesired consequences, such as poor growth of the engineered strain and/or poor expression of recombinant proteins, all of which can result in low production titer.

According to the present invention, the inventors have adapted the prokaryotic ssrA tagging system for use in Corynebacterium cells. The modified strain according to the present invention allows for the tunable degradation of one or more target proteins by adding the appropriate degradation tags to them. Different tags can be added to different protein targets allowing a differential control in degradation, both in terms of the extent of degradation and the use of multiple inducers in a single organism for parallel systems of control. In reporter systems, the competing requirements of signal detection and dynamical resolution can be balanced without the need for additional cloning procedures. This system has several advantages over previously described systems. The degradation is tunable and can be differentially tunable for multiple protein targets. The degradation tags are small and unlikely to interfere with protein function within the modified host cell. The size of the tag simplifies construction of tagged genes by PCR amplification or use of a tagging vector, and many genes can be tagged in parallel.

According to the current invention, the inventors demonstrated that the E. coli SspB adapter protein is fully compatible with the native Corynebacterium proteases, and is the first demonstration of using the ssrA tag system for targeted protein degradation in this genus. The inventors have validated that the general pattern of ssrA-tagged protein degradation based on several variants of the ssrA tag (such as the DAS+4 variant) are consistent between both E. coli and Corynebacterium. The inventors also have validated the use of the S. aureus trfA tag coupled with the TrfA adaptor protein in Corynebacterium as a replacement for the ssrA tag. The adaptor protein binding regions of the ssrA and trfA tags are vastly different and there is no cross-talk between the tag systems, potentially allowing the selective targeting of multiple proteins at different time points in the growth cycle. Finally, the inventors have demonstrated evolution of several alternatives to the DAS+4 ssrA tag by high-throughput screening of a broad, rationally designed library. The newly evolved ssrA tags demonstrate better dynamic range by reducing the background level of protein degradation in the absence of the SspB or TrfA adaptor protein, while still efficiently degrading the tagged protein after the adaptor is induced.

### Key Features

Certain key features of the present invention include the use of the ssrA and the trfA protein degradation tags. Both of these tags contain two sequence motifs. First, the recognition motif for the SsrA and TrfA adaptor proteins, contained in the first part of the sequence. Second, the 3 amino acids on the C-terminus containing a degradation motif recognized by cellular proteases such as ClpXP. The exact amino acid sequence of the three terminal residues dictates the rate of the degradation of the target protein. Previously, the DAS+4 tag proved to be essential in balancing the protein degradation rate. The inventors have identified novel tags, including the QPS, KPS, and DQA tags, that have better activity than the DAS+4 tag.

### Microbial Strain Construction

The adaptor proteins SspB and TrfA were integrated into the C. glutamicum chromosome as replacements for known IS elements. These sites were specifically chosen to minimize disruption of any native Corynebacterium metabolic pathways. The genes for the adaptors were placed at one of several integration sites and tested for their activity towards the reporter proteins. The sites used were ISCg2c, ISCg2e, and ISCg6c. Ultimately, site ISCg6c was chosen as the site with the best independent regulation. Two promoters were tested for the sspB adapter, specifically the C. glutamicum phosphate inducible promoter and the C. glutamicum optimized E. coli Tac promoter. The Tac promoter also contains the C. glutamicum optimized version of the lad repressor which was oriented in the opposite direction of the sspB adapter open reading frame. The trfA adapter was integrated and tested under the control of the Tac promoter and in the ISCg6c chromosomal locus. Genome integrations were performed as previously described using single cross-over knock-in based on flanking homology regions. The desired knock-in clones were selected via growth on kanamycin. A second single cross-over event was forced using sucrose selection and the resulting colonies were screened for the presence of desired mutants. Final C. glutamicum strains were free of any selection markers.

### Degradation Tags

To discover better performing degradation tags, the inventors screened a library of potential c-terminal amino acids supplanted onto the ssrA-DAS+4 tag. The library includes each of the possible combinations of amino acids in column 1+column 2+column 3 from Table 2 below. As shown by the Examples below, the current invention provides degradation tag variants that permit independent discrete control of both the initial level and inducible degradation rate of tagged proteins in Corynebacterium.

### EXAMPLES

### Methods and Materials:

The following strains were used in the examples below:
(1) C. glutamicum ATCC13032 was used as the base Corynebacterium strain for all experiments.
(2) C. glutamicum-lacI-sspB was recombinantly engineered with codon optimized E. coli sspB gene sequence chromosomally integrated under the control of the E. coli pTac promoter with repression provided by codon optimized E. coli lad (a lac repressor).
(3) C. glutamicum-lacI-trfA was recombinantly engineered with codon optimized E. coli trfA gene sequence chromosomally integrated under the control of the E. coli pTac promoter with repression provided by codon optimized E. coli lad.
(4) E. coli 10G was used as the standard cloning strain.

The following plasmids were used in the examples below:

CgDVK-mCherry denotes a Corynebacterium shuttle vector containing the reporter gene (mCherry) under the control of either a pSOD promoter (a strong promoter) or a Min5 promoter (a weak promoter). All plasmids containing modified degradation tags were constructed by modifying the c-terminus of the mCherry reporter gene on this plasmid backbone.

Transformation. Corynebacterium strains were transformed with the plasmid expressing mCherry or mCherry-tag, with the tag sequences and names outlined in Table 1 below. Transformations were performed using standard electroporation protocols. The transformants were selected on Caso-Kan25.

TABLE 1
Names and amino acid sequence of the selective
degradation tags tested. The tags tested were
added to the C-terminus of the reporter protein
and were followed by the stop codon. Tag
sequence is shown in amino acid format.
Sequences shown in italics represent adaptor
protein (SspB or TrfA) binding regions.
Sequences shown in bold represent regions
recognized by the cellular Clp proteases.
Sequences for ssrA-LAA and trfA-VAA
represent WT adaptor/protease recognition
sequence pairs in the native hosts.

| Tag Name | Tag Sequence | SEQ ID NO. |
|---|---|---|
| mCherry (not tagged) | N/A | |
| ssrA-LAA | AANDENYALAA | SEQ ID NO. 8 |
| (wild-type E. coli) | | |
| ssrA-DAS | AANDENYADAS | SEQ ID NO. 10 |
| ssrA-DAS + 4 | AANDENYSENYADAS | SEQ ID NO. 12 |
| ssrA-DQP + 4 | AANDENYSENYADQP | SEQ ID NO. 14 |
| ssrA-KPS + 4 | AANDENYSENYAKPS | SEQ ID NO. 16 |
| ssrA-DGA + 4 | AANDENYSENYADGA | SEQ ID NO. 18 |
| ssrA-DGS + 4 | AANDENYSENYADGS | SEQ ID NO. 20 |
| trfA-VAA | GKSNNNFAVAA | SEQ ID NO. 22 |
| (wild-type S. aureus) | | |
| trfA-DAS | GKSNNNFADAS | SEQ ID NO. 24 |
| trfA-DAS + 4 | GKSNNNFSNNFADAS | SEQ ID NO. 26 |
| trfA-DQP + 4 | GKSNNNFSNNFADQP | SEQ ID NO. 28 |
| trfA-KPS + 4 | GKSNNNFSNNFAKPS | SEQ ID NO. 30 |
| trfA-DGA + 4 | GKSNNNFSNNFADGA | SEQ ID NO. 32 |
| trfA-DGS + 4 | GKSNNNFSNNFADGS | SEQ ID NO. 34 |

Strains containing either the sspB or trfA adaptor sequence on the chromosome were created using standard homologous recombination techniques using a suicide vector containing a Kan positive selection and a sacB negative selection markers, ultimately resulting in a marker-less modification. Protocols for plasmid transformation and mCherry reporter assays using these engineered strains is the same as using wild-type Corynebacterium.

DAS+4 mutant library construction and screening. Mutants were created using Gibson Assembly by amplifying the pSOD-mCherry-ssrA-DAS region from the pCBMK-mCherry-DAS+4 plasmid. The mutations to create the libraries were introduced into the reverse primer. The amplified region was inserted into the pZ8 vector. The product of the Gibson Assembly was electroporated directly into the C. glutamicum lacI-sspB strain. The resulting colonies were selected on Caso+Kan 25 selection again.

High-throughput colony selection. Individual colonies from selection plates were picked via machine vision on an automated liquid handler, inoculated into 600 μl of BHI-Kan25 and allowed to grow overnight at 30° C. Overnight cultures were further diluted into either BHI-Kan25 or CGXII-Kan25 and induced with IPTG, where required for either adaptor protein or reporter protein synthesis. Choice of assay medium did not significantly impact final experimental outcome, although the background fluorescence of BHI was significantly higher than that of CGXII. Following ˜48 hours, the aliquots from cultures were diluted 1:20 into 200 μl of water and the OD650 and mCherry fluorescence was measured at the excitation-emission wavelengths of 585 nm-615 nm.

Two 96-well plates were picked from each of the 18 created libraries and initially cultured in BHI. Once the libraries have grown, they were seeded into CGXII media with or without IPTG to induce the expression of SspB adaptor protein. Fluorescence and absorbance measurements for each culture were taken after approximately 48 hours of growth. The ratio of fluorescence of uninduced to induced cultures was used to select the initial positive hits from the assay.

Initial hits were validated a second time in a 48-well plate assay, following the same protocol as above, in a BioLector microbioreactor system (m2p-labs GmbH, Germany) in order to obtain detailed growth curves and expression patterns. Plasmids were isolated from the eight best performing isolates. The regions encoding the mCherry and the new DAS tag variants were sequenced to determine the final changes. Additionally, the top hits were validated a second time in a larger culture volume following the same protocols.

### Results:

Validation of SspB Functionality in C. glutamicum

The ability to selectively degrade target proteins hinges on the ability of SspB adaptor protein to bind the ssrA sequence and help initiate degradation of the target protein via cellular Clp protease complex. Given that the SspB/ssrA system was isolated from E. coli it was essential to first determine its functionality in a heterologous host.

As a result, SspB was integrated into the chromosome of C. glutamicum cells under the control of an inducible promoter (more precisely, the coupling of a constitutive promoter pTac controlled by the inducible lac repressor lad), in order to have on/off control of its activity. A degradation tag (wild-type ssrA degradation tag and synthetic variants thereof, the sequences of which are provided in Table 1) was added to an mCherry reporter and introduced into the host. The resulting data shows that the E. coli SspB adaptor protein maintains activity and ability to increase selective degradation of targeted protein in C. glutamicum.

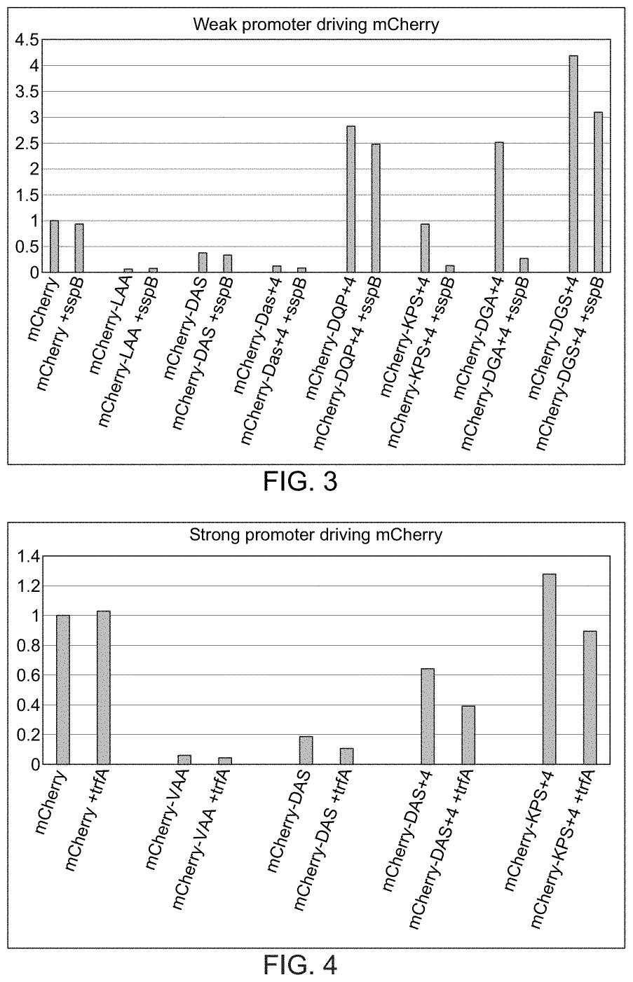

Specifically, referring to FIG. 2, it can be seen that when the target protein (in this case, mCherry) was tagged by ssrA (mCherry-LAA) and driven by a strong promoter (pSOD), almost all the target proteins were proteolyzed. When SspB was induced (mCherry-LAA+sspB), even more target proteins were proteolyzed. In the case when the mCherry reporter was driven by a weak promoter (Min5), the degradation rate appeared comparable between whether SspB was induced or not (FIG. 3).

Validation of TrfA Functionality in C. glutamicum

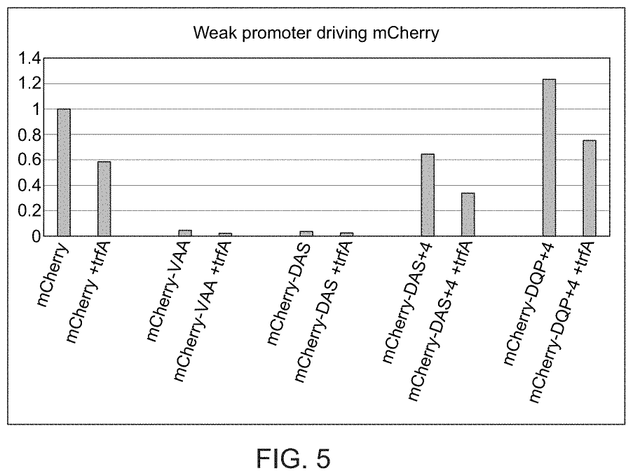

Unlike the SspB/ssrA system, which has been extensively studied in several hosts, the activity of TrfA adaptor protein [SEQ ID NO. 4] has been demonstrated only in the native host S. aureus. Additionally, little has been studied about the efficiency of TrfA-promoted protein degradation when using modified Clp recognition sequences. Prior to use in any application, extensive validation of TrfA activity in any host was necessary. TrfA functionality was evaluated using the SspB/ssrA system as both a guide and a baseline for minimal required activity. FIG. 4 and FIG. 5 demonstrate that the TrfA system behaves in a similar manner compared to the SspB system in C. glutamicum when using the wild type protease recognition tag trfA [SEQ ID NO. 22]. Because the two systems function similarly yet, as shown further below, have distinct properties regarding the efficiency of protein degradation kinetics, the TrfA system and the SspB system can be integrated into the same host as two orthogonal systems, which in turn provide a mechanism to fine-tune protein degradation in biosynthetic production methods that require control of at least two different essential genes.

### Screening of Better Performing Tag Variants

The C-terminus amino acid composition of a protein plays a large role in whether the said protein is recognized and degraded by cellular proteases. The native ssrA and trfA protein degradation tags feature, respectively, amino acids LAA and VAA as the terminal amino acids added to the target protein. Both of those sequences result in rapid degradation of the target protein, even without an adaptor present (see mCherry-LAA in FIGS. 2-3 ad mCherry-VAA in FIGS. 4-5). Several variant sequences have been previously explored, such as the DAS and the DAS+4 sequences tested in this work. However, as demonstrated here, neither of those tags are optimal for the application of induced protein degradation because the addition of either of those tags greatly reduces the amount of the target protein in the cells, even before induction of the adaptor protein. If the target protein is an essential protein for cell growth, its reduction of over 50% compared to the baseline presence could be highly detrimental to cell health.

Accordingly, the next step was to screen variants with modified protease recognition sequences (last 3 amino acids of the C-terminal region) compared to the wild-type, that show better performance, i.e., tags that cause minimal protein degradation in the absence of the adaptor protein but high level of degradation after induced expression of the adaptor protein.

To do this, the inventors set out to screen rationally designed combinatorial libraries of Clp protease recognition sequences. This screen was performed directly into C. glutamicum given that was the final host organism. The amino acids and corresponding DNA sequences are shown in Tables 2 and 3 below.

TABLE 2
Amino acid combinations
incorporated into a
randomized library to select
better performing variants of the
ssrA-DAS+4tag/trfA-DAS+4
tag. Positions 1, 2, and 3
correspond to amino acids
D, A, and S of the
DAS tag, respectively.
Amino acid by position

| 1 | 2 | 3 |
|---|---|---|
| K | E | P |
| M | A | A |
| Q | Y | S |
| P | S | K |
| N | K | N |
| H | N | M |
| D | M | I |
| | I | |
| | Q | |
| | P | |

TABLE 3
Breakdown of each compressed library used for selections of novel
ssrA-tag variants/trfA-tag variants. Both degenerate IUPAC names
for the nucleotides as well as single letter amino acids are shown
for each library screened at each of the three randomized positions.

| Lib | Nucleotide Sequence | | | Possible amino acids | | |
|---|---|---|---|---|---|---|
| # | 1 | 2 | 3 | 1 | 2 | 3 |
| 1 | AWG | KMT | BCG | K, M | E, A, Y, S | P, A, S |
| 2 | AWG | KMT | AWS | K, M | E, A, Y, S | K, N, M, I |
| 3 | AWG | AWS | BCG | K, M | K, N, M, I | P, A, S |
| 4 | AWG | AWS | AWS | K, M | K, N, M, I | K, N, M, I |
| 5 | AWG | CMA | BCG | K, M | Q, P | P, A, S |
| 6 | AWG | CMA | AWS | K, M | Q, P | K, N, M, I |
| 7 | CMA | KMT | BCG | Q, P | E, A, Y, S | P, A, S |
| 8 | CMA | KMT | AWS | Q, P | E, A, Y, S | K, N, M, I |
| 9 | CMA | AWS | BCG | Q, P | K, N, M, I | P, A, S |
| 10 | CMA | AWS | AWS | Q, P | K, N, M, I | K, N, M, I |
| 11 | CMA | CMA | BCG | Q, P | Q, P | P, A, S |
| 12 | CMA | CMA | AWS | Q, P | Q, P | K, N, M, I |
| 13 | VAT | KMT | BCG | N, H, D | E, A, Y, S | P, A, S |
| 14 | VAT | KMT | AWS | N, H, D | E, A, Y, S | K, N, M, I |
| 15 | VAT | AWS | BCG | N, H, D | K, N, M, I | P, A, S |
| 16 | VAT | AWS | AWS | N, H, D | K, N, M, I | K, N, M, I |
| 17 | VAT | CMA | BCG | N, H, D | Q, P | P, A, S |
| 18 | VAT | CMA | AWS | N, H, D | Q, P | K, N, M, I |

After transformation of the libraries into the host, the resulting transformants were screened for both high levels of reporter protein when SspB/TrfA was not present as well as low levels of the reporter protein after SspB/TrfA induction. The results and the top hits from this screen are shown in Table 4. The top hits from the initial screen were cherry-picked and re-confirmed at larger scale. Finally, the functionality of the best hits were re-screened coupled with both ssrA and trfA recognition sequences across multiple promoters driving the expression of the reporter gene.

TABLE 4
New tag sequences as determined b sequencing of eight selected
plasmids from the screen, including the top three performing clones
from the screen. In the table, “Name” identifies the mutant by plate
location; “Sequence” refers to the amino acid sequence of the DAS
variant, assessed by sequencing of the plasmid; “Screen 1 ratio”
refers to the ratio of the mCherry fluorescence under uninduced/
induced conditions during the initial screen; “Hitpick ratio” refers
to the ratio of mCherry fluorescence after the top performing
cultures from screen 1 were cherrypicked and re-screened
to verify their activity.

| Name | Sequence | Screen1 ratio | Hitpick ratio |
|---|---|---|---|
| DAS+4 | DAS | ~5 | ~5 |
| 17B_F10 | DQP* | 13.059 | 7.446 |
| 5A_E10 | KPS* | 10.270 | 9.452 |
| 17A_E6 | DQA* | 9.247 | 8.016 |
| 3A_6B | KNP* | 9.231 | 3.142 |
| 11B_C9 | QPS* | 9.224 | 7.804 |
| 3A_D7 | MKP* | 7.793 | 2.588 |
| 17B | DQS* | 7.542 | 5.695 |
| 17A_F12 | HPP* | 6.009 | 7.194 |

Ultimately, several of the selected tags were found to give better uninduced/induced response curves than the previously designed DAS+4 tag, especially the KPS+4 and DGA+4 tags (FIGS. 2-5).

Specifically, variants showing desirable modulating effects on protein degradation in the SspB system was shown in FIG. 2 and FIG. 3. These data collectively provide evidence that the efficiency with which SspB is able to promote protein degradation varies based on both the degradation tag and the strength of the promoter driving the expression of the target gene. This discovery was crucial as premature degradation of essential proteins can cause cell death. A desirable pairing of degradation tag and adaptor protein includes the case when degradation is minimal when the target protein is tagged, and significant degradation only takes place upon induction of the adapter protein. When comparing the ratio of the target protein signal (in the form of normalized fluorescence strength here) prior to and after induction of SspB, one can see that the ssrA-KPS+4 tag [SEQ ID NO. 16] (cf. mCherry-KPS+4 against mCherry-KPS+4+sspB) achieves this objective. More specifically, one can see that in the case when the tagged mCherry was driven by a strong promoter, there was some degradation (˜7%) prior to induction of SspB, but significant degradation was observed after induction of SspB (˜32%). See FIG. 2. The effect was even more pronounced when the tagged mCherry was driven by a weak promoter. Again, there was some degradation (˜7%) prior to induction of SspB, but after SspB induction, degradation increased dramatically (˜93%).

Similarly, the KPS+4 tag [SEQ ID NO. 30] showed desirable modulating effects on protein degradation in the TrfA system, regardless of whether the tagged mCherry was driven by a stronger promoter (FIG. 4) or a weak promoter (FIG. 5).

### Limited Cross-Talk Between the SspB System and the TrfA System

Given the above data collectively show that the SspB and the TrfA systems each recognize a distinct signal sequence and both retain activity in C. glutamicum host cells, the inventors proceeded to investigate whether there is cross-talk between the two adaptor proteins. If the two TrfA and SspB systems are able to function individually, without causing degradation of orthogonally tagged proteins, it would allow precise temporal control over multiple cell functions. For example, it would be possible to trigger degradation of a first protein that plays a role in the fitness of the cell once a desired biomass has been reached using the first of the tags. Subsequently, degradation of a second target protein could be triggered at a later time point, once a sufficient amount of an intermediate has been reached. This temporal control is important for truly fine-tuning the optimal production conditions for biosynthesizing a desired product. This type of control is only possible if the two tags are truly orthogonal, and do not cross react with the recognition sequence of the other tag. This was tested by introducing an ssrA-tagged reporter into a strain containing the TrfA adaptor protein and a trfA-tagged reporter into a strain containing the SspB adaptor protein. In addition to the WT trfA and ssrA sequences, several of the engineered sequences were also tested. The data presented in Table 5 show insignificant cross-talk between the two protein degradation systems, indicating it is possible to use the two in parallel.

TABLE 5
TrfA and SspB specificity. TrfA and SspB adapters
were screened for crosstalk and found to be orthogonal.
Greater degradation of reporter was observed when the
tag-type matched the host adapter. Degradation is
reported as a percentile of reporter concentration before
adapter induction. Effects were similar for both strong
and weak expression of the tagged reporter gene.

| Promoter | Host | Reporter | |
|---|---|---|---|
| strength | adapter | tag | % degradation |
| Weak | sspB | ssrA | 35 |
| Weak | sspB | trfA | 15 |
| Strong | sspB | ssrA | 55 |
| Strong | sspB | trfA | 32 |
| Weak | trfA | trfA | 48 |
| Weak | trfA | ssrA | 20 |
| Strong | trfA | trfA | 39 |
| Strong | trfA | ssrA | 27 |

### STATEMENT OF INDUSTRIAL APPLICABILITY/TECHNICAL FIELD

This disclosure has applicability in the food, medicinal, and pharmacological industries. This disclosure relates generally to a method for the strategic control of protein degradation in modified microbial strains. Such modifications lead to enhanced production yield of compounds of interest, extended duration of optimized compounds in a cell environment all while limiting the long-term damage to the modified cellular host.

Sequences of Interest:
SEQ ID NO. 1 - WT SspB DNA sequence:
ATGGATTTGTCGCAACTCACTCCTCGGCGGCCTTATCTGCTGCGTGCTTTTTACGAGTGGCTGCTTGATAACCAGTT
GACGCCTCATCTCGTAGTGGATGTAACCCTGCCCGGAGTCCAGGTCCCAATGGAATACGCTCGTGACGGCCAGAT
CGTTTTGAATATTGCTCCACGTGCGGTTGGTAACCTTGAACTTGCTAACGATGAAGTCCGTTTTAATGCCCGCTTCG
GTGGCATCCCGCGCCAAGTCTCAGTACCTCTCGCCGCAGTTCTGGCCATTTATGCCCGCGAAAATGGCGCAGGCAC
TATGTTTGAGCCGGAGGCAGCCTATGACGAGGATACCTCTATTATGAATGATGAAGAGGCGTCCGCCGATAATGA
GACGGTAATGTCAGTTATTGATGGTGACAAACCGGATCACGACGATGACACTCATCCAGATGACGAGCCCCCGCA
ACCACCACGCGGCGGACGACCGGCCTTGCGAGTGGTCAAATAA
SEQ ID NO. 2 - WT SspB amino acid sequence:
MDLSQLTPRRPYLLRAFYEWLLDNQLTPHLVVDVTLPGVQVPMEYARDGQIVLNIAPRAVGNLELANDEVRFNARFG
GIPRQVSVPLAAVLAIYARENGAGTMFEPEAAYDEDTSIMNDEEASADNETVMSVIDGDKPDHDDDTHPDDEPPQPP
RGGRPALRVVK
SEQ ID NO. 3 WT TrfA DNA sequence:
ATGAGAATAGAACGAGTAGATGATACAACTGTAAAATTGTTTATAACATATAGCGATATCGAGGCCCGTGGATTTA
GTCGTGAAGATTTATGGACAAATCGCAAACGTGGCGAAGAGTTCTTTTGGTCAATGATGGATGAAATTAACGAAG
AAGAAGATTTTGTTGTAGAAGGTCCATTATGGATTCAAGTACATGCCTTTGAAAAAGGTGTCGAAGTCACAATTTC
TAAATCTAAAAATGAAGATATGATGAATATGTCTGATGATGATGCAACTGATCAATTTGATGAACAAGTTCAAGAA
TTGTTAGCTCAAACATTAGAAGGTGAAGATCAATTAGAAGAATTATTCGAGCAACGAACAAAAGAAAAAGAAGCT
CAAGGTTCTAAACGTCAAAAGTCCTCAGCACGTAAAAATACAAGAACAATCATTGTGAAATTTAACGATTTAGAAG
ATGTTATTAATTATGCATATCATAGCAATCCAATAACTACAGAGTTTGAAGATTTGTTATATATGGTTGATGGTACT
TATTATTATGCTGTACATTTTGATAGTCATGTTGATCAAGAAGTCATTAATGATAGTTACAGTCAATTGCTTGAATTT
GCATATCCAACAGACAGAACAGAAGTTTATTTAAATGACTATGCTAAAATAATTATGAGTCATAACGTAACAGCTC
AAGTTCGACGTTATTTTCCAGAGACAACTGAATAA
SEQ ID NO. 4 WT TrfA amino acid sequence:
MRIERVDDTTVKLFITYSDIEARGFSREDLWTNRKRGEEFFWSMMDEINEEEDFVVEGPLWIQVHAFEKGVEVTISKSK
NEDMMNMSDDDATDQFDEQVQELLAQTLEGEDQLEELFEQRTKEKEAQGSKRQKSSARKNTRTIIVKFNDLEDVINY
AYHSNPITTEFEDLLYMVDGTYYYAVHFDSHVDQEVINDSYSQLLEFAYPTDRTEVYLNDYAKIIMSHNVTAQVRRYFPE
TTE
SEQ ID NO. 5 mCherry DNA sequence:
ATGGTCAGCAAAGGTGAGGAAGATAACATGGCAATTATTAAGGAATTTATGCGCTTTAAAGTTCATATGGAGGGT
TCGGTTAATGGTCACGAGTTTGAAATCGAAGGCGAGGGAGAGGGTCGTCCATATGAGGGCACTCAGACCGCTAA
ACTCAAAGTAACGAAGGGCGGTCCGCTTCCGTTCGCCTGGGACATTCTTAGCCCACAATTTATGTACGGTTCTAAG
GCTTACGTGAAACATCCCGCCGACATTCCAGACTATCTGAAACTGTCATTCCCTGAAGGTTTCAAATGGGAACGTG
TGATGAACTTCGAGGATGGCGGTGTAGTGACCGTCACCCAAGACAGCTCCCTTCAGGACGGCGAATTCATTTACA
AAGTAAAGTTGCGCGGAACCAATTTCCCCTCCGATGGCCCAGTAATGCAAAAGAAAACGATGGGATGGGAAGCAT
CCTCGGAGCGTATGTATCCGGAGGACGGCGCTTTGAAAGGAGAAATCAAACAGCGCCTTAAGTTGAAAGACGGT
GGCCATTACGACGCGGAGGTCAAAACGACCTATAAGGCTAAGAAACCTGTTCAGCTGCCGGGTGCATATAACGTC
AACATTAAGTTGGACATCACTTCCCATAACGAGGATTACACCATCGTGGAACAGTATGAACGTGCAGAGGGCCGC
CACAGCACTGGCGGCATGGATGAGCTTTACAAGTAA
SEQ ID NO. 6 mCherry amino acid sequence:
MVSKGEEDNMAIIKEFMRFKVHMEGSVNGHEFEIEGEGEGRPYEGTQTAKLKVTKGGPLPFAWDILSPQFMYGSKAY
VKHPADIPDYLKLSFPEGFKWERVMNFEDGGVVTVTQDSSLQDGEFIYKVKLRGTNFPSDGPVMQKKTMGWEASSER
MYPEDGALKGEIKQRLKLKDGGHYDAEVKTTYKAKKPVQLPGAYNVNIKLDITSHNEDYTIVEQYERAEGRHSTGGMD
ELYK
SEQ ID NO. 7 WT ssrA tag DNA sequence:
GCCGCTAATGACGAAAACTATGCGCTTGCAGCA
SEQ ID NO. 8 WT ssrA tag amino acid sequence:
AANDENYALAA
SEQ ID NO. 9 ssrA-DAS variant tag DNA sequence:
GCGGCGAATGATGAGAATTACGCTGACGCTTCG
SEQ ID NO. 10 ssrA-DAS variant tag amino acid sequence:
AANDENYADAS
SEQ ID NO. 11 ssrA-DAS + 4 variant tag DNA sequence:
GCGGCGAATGATGAGAATTACAGCGAGAACTACGCTGACGCTTCG
SEQ ID NO. 12 ssrA-DAS + 4 variant tag amino acid sequence:
AANDENYSENYADAS
SEQ ID NO. 13 ssrA-DQP + 4 variant tag DNA sequence:
GCGGCGAATGATGAGAATTACAGCGAGAACTACGCTGATCAACCG
SEQ ID NO. 14 ssrA-DQP + 4 variant tag amino acid sequence:
AANDENYSENYADQP
SEQ ID NO. 15 mCherry-ssrA-KPS + 4 DNA:
GCGGCGAATGATGAGAATTACAGCGAGAACTACGCTAAGCCATCG
SEQ ID NO. 16 ssrA-KPS + 4 variant tag amino acid sequence:
AANDENYSENYAKPS
SEQ ID NO. 17 ssrA-DGA + 4 variant tag DNA sequence:
GCGGCGAATGATGAGAATTACAGCGAGAACTACGCTGATGGCGCG
SEQ ID NO. 18 ssrA-DGA + 4 variant tag amino acid sequence:
AANDENYSENYADGA
SEQ ID NO. 19 ssrA-DGS + 4 variant tag DNA sequence:
GCGGCGAATGATGAGAATTACAGCGAGAACTACGCTGATGGCTCG
SEQ ID NO. 20 ssrA-DGS + 4 variant tag amino acid sequence:
AANDENYSENYADGS
SEQ ID NO. 21 WT trfA tag DNA sequence:
GGCAAATCAAACAATAATTTCGCAGTAGCTGCC
SEQ ID NO. 22 WT trfA tag amino acid sequence:
GKSNNNFAVAA
SEQ ID NO. 23 trfA-DAS variant tag DNA sequence:
GGCAAATCAAACAATAATTTCGCAGACGCTTCG
SEQ ID NO. 24 trfA-DAS variant tag amino acid sequence:
GKSNNNFADAS
SEQ ID NO. 25 trfA-DAS + 4 variant tag DNA sequence:
GGCAAATCAAACAATAATTTCAGCAACAATTTCGCAGACGCTTCG
SEQ ID NO. 26 trfA-DAS + 4 variant tag amino acid sequence:
GKSNNNFSNNFADAS
SEQ ID NO. 27 trfA-DQP + 4 variant tag DNA sequence:
GGCAAATCAAACAATAATTTCAGCAACAATTTCGCAGATCAACCG
SEQ ID NO. 28 trfA-DQP + 4 variant tag amino acid sequence:
GKSNNNFSNNFADQP
SEQ ID NO. 29 trfA-KPS + 4 variant tag DNA sequence:
GGCAAATCAAACAATAATTTCAGCAACAATTTCGCAAAGCCATCG
SEQ ID NO. 30 trfA-KPS + 4 variant tag amino acid sequence:
GKSNNNFSNNFAKPS
SEQ ID NO. 31 trfA-DGA + 4 variant tag DNA sequence:
GGCAAATCAAACAATAATTTCAGCAACAATTTCGCAGATGGCGCG
SEQ ID NO. 32 trfA-DGA + 4 variant tag amino acid sequence:
GKSNNNFSNNFADGA
SEQ ID NO. 33 trfA-DGS + 4 variant tag DNA sequence:
GGCAAATCAAACAATAATTTCAGCAACAATTTCGCAGATGGCTCG
SEQ ID NO. 34 trfA-DGS + 4 variant tag amino acid sequence:
GKSNNNFSNNFADGS
SEQ ID NO. 35 ssrA-DQA + 4 variant tag amino acid sequence:
AANDENYSENYADQA
SEQ ID NO. 36 ssrA-QPS + 4 variant tag amino acid sequence:
AANDENYSENYAQPS
SEQ ID NO. 37 ssrA-HPP + 4 variant tag amino acid sequence:
AANDENYSENYAHPP
SEQ ID NO. 38 ssrA-DQS + 4 variant tag amino acid sequence:
AANDENYSENYADQS
SEQ ID NO. 39 ssrA-KNP + 4 variant tag amino acid sequence:
AANDENYSENYAKNP
SEQ ID NO. 40 ssrA-MKP + 4 variant tag amino acid sequence:
AANDENYSENYAMKP
SEQ ID NO. 41 ssrA-DQA variant tag amino acid sequence:
AANDENYADQA
SEQ ID NO. 42 ssrA-QPS variant tag amino acid sequence:
AANDENYAQPS
SEQ ID NO. 43 ssrA-HPP variant tag amino acid sequence:
AANDENYAHPP
SEQ ID NO. 44 ssrA-DQS variant tag amino acid sequence:
AANDENYADQS
SEQ ID NO. 45 ssrA-KNP variant tag amino acid sequence:
AANDENYAKNP
SEQ ID NO. 46 ssrA-MKP variant tag amino acid sequence:
AANDENYAMKP
SEQ ID NO. 47 trfA-DQA + 4 variant tag amino acid sequence:
GKSNNNFSNNFADQA
SEQ ID NO. 48 trfA-QPS + 4 variant tag amino acid sequence:
GKSNNNFSNNFAQPS
SEQ ID NO. 49 trfA-HPP + 4 variant tag amino acid sequence:
GKSNNNFSNNFAHPP
SEQ ID NO. 50 trfA-DQS + 4 variant tag amino acid sequence:
GKSNNNFSNNFADQS
SEQ ID NO. 51 trfA-KNP + 4 variant tag amino acid sequence:
GKSNNNFSNNFAKNP
SEQ ID NO. 52 trfA-MKP + 4 variant tag amino acid sequence:
GKSNNNFSNNFAMKP
SEQ ID NO. 53 trfA-DQA variant tag amino acid sequence:
GKSNNNFADQA
SEQ ID NO. 54 trfA-QPS variant tag amino acid sequence:
GKSNNNFAQPS
SEQ ID NO. 55 trfA-HPP variant tag amino acid sequence:
GKSNNNFAHPP
SEQ ID NO. 56 trfA-DQS variant tag amino acid sequence:
GKSNNNFADQS
SEQ ID NO. 57 trfA-KNP variant tag amino acid sequence:
GKSNNNFAKNP
SEQ ID NO. 58 trfA-MKP variant tag amino acid sequence:
GKSNNNFAMKP

What is claimed is:

1. A Corynebacterium species host cell recombinantly engineered to express a first degradation tag, wherein said first degradation tag comprises an adaptor binding region, an optional spacer region, and a protease recognition region; wherein said adaptor binding region of the first degradation tag specifically binds a TrfA adaptor protein comprising an amino acid sequence having at least 90% sequence identity to SEQ ID NO. 4.

2. The host cell of claim 1, wherein said protease recognition region of the first degradation tag allows a target protein tagged by the first degradation tag to be degraded by a protease selected from the group consisting of ClpCP, ClpXP and ClpAP when the TrfA adaptor protein is present.

3. The host cell of claim 1, wherein said first degradation tag comprises the amino acid sequence of SEQ ID NO. 30 or SEQ ID NO. 32.

4. The host cell of claim 1, wherein said first degradation tag comprises the amino acid sequence of SEQ ID NO. 28, SEQ ID NO. 34, SEQ ID NO. 47, SEQ ID NO. 48, SEQ ID NO. 49, SEQ ID NO. 50, SEQ ID NO. 51, SEQ ID NO. 52, SEQ ID NO. 53, SEQ ID NO. 54, SEQ ID NO. 55, SEQ ID NO. 56, SEQ ID NO. 57, or SEQ ID NO. 58.

5. The host cell of claim 1, wherein said first degradation tag comprises the amino acid sequence of SEQ ID NO. 24 or SEQ ID NO. 26.

6. The host cell of claim 1 further recombinantly engineered to express a second degradation tag, wherein said second degradation tag comprises an adaptor binding region, an optional spacer region, and a protease recognition region; wherein said adaptor binding region of the second degradation tag specifically binds a SspB adaptor protein comprising an amino acid sequence having at least 90% sequence identity to SEQ ID NO. 2.

7. The host cell of claim 6, wherein said protease recognition region of the second degradation tag allows a target protein tagged by the second degradation tag to be degraded by a protease selected from the group consisting of ClpCP, ClpXP and ClpAP when the SspB adaptor protein is present.

8. The host cell of claim 6, wherein said second degradation tag comprises the amino acid sequence of SEQ ID NO. 16 or SEQ ID NO. 18.

9. The host cell of claim 1, wherein said first degradation tag comprises the amino acid sequence of SEQ ID NO. 14, SEQ ID NO. 20, SEQ ID NO. 35, SEQ ID NO. 36, SEQ ID NO. 37, SEQ ID NO. 38, SEQ ID NO. 39, SEQ ID NO. 40, SEQ ID NO. 41, SEQ ID NO. 42, SEQ ID NO. 43, SEQ ID NO. 44, SEQ ID NO. 45, or SEQ ID NO. 46.

10. The host cell of claim 1, wherein said first degradation tag comprises the amino acid sequence of SEQ ID NO. 10 or SEQ ID NO. 12.

11. A method of controlling the degradation of a first target protein in a Corynebacterium species host cell, wherein said host cell has been recombinantly engineered to express a first degradation tag and to produce a first product via a first heterologous biosynthetic pathway, the method comprising:
expressing the first degradation tag adapted to tag the first target protein; growing the host cell until a desired growth rate is reached; and
inducing the expression of a TrfA adaptor protein, wherein the TrfA adaptor protein comprises an amino acid sequence having at least 90% sequence identity to SEQ ID NO. 4;
wherein the amount of the first target protein present in the host cell decreases by at least about 20% after the expression of the TrfA adaptor protein is induced, and wherein such decrease is caused by degradation by a protease selected from the group consisting of ClpCP, ClpXP and ClpAP.

12. The method of claim 11, wherein said first target protein is an essential protein for the growth of the host cell.

13. The method of claim 11, wherein the presence of said first target protein functions as a negative feedback in said first heterologous biosynthetic pathway for producing said first product.

14. The method of claim 11, wherein said first target protein metabolizes the first product, thereby reducing the collectible amount of said first product.

15. The method of claim 11, wherein the expression of the TrfA adaptor protein is induced by a temperature change, a pH change, exposure to light and/or by changing the level of a given molecule within the host cell.

16. The method of claim 11, wherein the last three amino acid sequence of the C-terminus of said first degradation tag is selected from the group consisting of DQP, KPS, DGA, DGS, DQA, KNP, QPS, MKP, DQS, and HPP.

17. The method of claim 11, wherein said host cell has been further recombinantly engineered to express a second degradation tag and to produce a second product via a second heterologous biosynthetic pathway, the method further comprising:
expressing the second degradation tag to tag the second target protein; growing the host cell until a desired growth rate is reached; and
inducing the expression of an SspB adaptor protein, wherein the SspB adaptor protein comprises an amino acid sequence having at least 90% sequence identity to SEQ ID NO. 2;
wherein the amount of the second target protein present in the host cell decreases by at least about 20% after the expression of the SspB adaptor protein is induced, and wherein such decrease is caused by degradation by a protease selected from the group consisting of ClpCP, ClpXP and ClpAP.

18. The method of claim 17, wherein said second target protein is an essential protein for the growth of the host cell.

19. The method of claim 17, wherein the presence of said second target protein functions as a negative feedback in said second heterologous biosynthetic pathway for producing said second product.

20. The method of claim 17, wherein said second target protein metabolizes the second product, thereby reducing the collectible amount of said second product.

21. The method of claim 17, wherein the expression of the SspB adaptor protein is induced by a temperature change, a pH change, exposure to light and/or by changing the level of a given molecule within the host cell.

22. The method of claim 17, wherein the last three amino acid sequence of the C-terminus of said second degradation tag is selected from the group consisting of DQP, KPS, DGA, DGS, DQA, KNP, QPS, MKP, DQS, and HPP.


SEQUENCE LISTING (58 sequences)

SEQ ID NO: 1 - DNA, 498 bp, Escherichia coli
atggatttgtcgcaactcactcctcggcggccttatctgctgcgtgctttttacgagtggctgcttgataaccagttgacgcctcatctcgtagtggatgtaaccctgcccggagtccaggtcccaatggaatacgctcgtgacggccagatcgttttgaatattgctccacgtgcggttggtaaccttgaacttgctaacgatgaagtccgttttaatgcccgcttcggtggcatcccgcgccaagtctcagtacctctcgccgcagttctggccatttatgcccgcgaaaatggcgcaggcactatgtttgagccggaggcagcctatgacgaggatacctctattatgaatgatgaagaggcgtccgccgataatgagacggtaatgtcagttattgatggtgacaaaccggatcacgacgatgacactcatccagatgacgagcccccgcaaccaccacgcggcggacgaccggccttgcgagtggtcaaataa

SEQ ID NO: 2 - protein, 165 aa, Escherichia coli [sequence omitted]

SEQ ID NO: 3 - DNA, 720 bp, Staphylococcus aureus
atgagaatagaacgagtagatgatacaactgtaaaattgtttataacatatagcgatatcgaggcccgtggatttagtcgtgaagatttatggacaaatcgcaaacgtggcgaagagttcttttggtcaatgatggatgaaattaacgaagaagaagattttgttgtagaaggtccattatggattcaagtacatgcctttgaaaaaggtgtcgaagtcacaatttctaaatctaaaaatgaagatatgatgaatatgtctgatgatgatgcaactgatcaatttgatgaacaagttcaagaattgttagctcaaacattagaaggtgaagatcaattagaagaattattcgagcaacgaacaaaagaaaaagaagctcaaggttctaaacgtcaaaagtcctcagcacgtaaaaatacaagaacaatcattgtgaaatttaacgatttagaagatgttattaattatgcatatcatagcaatccaataactacagagtttgaagatttgttatatatggttgatggtacttattattatgctgtacattttgatagtcatgttgatcaagaagtcattaatgatagttacagtcaattgcttgaatttgcatatccaacagacagaacagaagtttatttaaatgactatgctaaaataattatgagtcataacgtaacagctcaagttcgacgttattttccagagacaactgaataa

SEQ ID NO: 4 - protein, 239 aa, Staphylococcus aureus
MRIERVDDTTVKLFITYSDIEARGFSREDLWTNRKRGEEFFWSMMDEINEEEDFVVEGPLWIQVHAFEKGVEVTISKSKNEDMMNMSDDDATDQFDEQVQELLAQTLEGEDQLEELFEQRTKEKEAQGSKRQKSSARKNTRTIIVKFNDLEDVINYAYHSNPITTEFEDLLYMVDGTYYYAVHFDSHVDQEVINDSYSQLLEFAYPTDRTEVYLNDYAKIIMSHNVTAQVRRYFPETTE

SEQ ID NO: 5 - DNA, 711 bp, Unknown
  Discosoma sea anemone
atggtcagcaaaggtgaggaagataacatggcaattattaaggaatttatgcgctttaaagttcatatggagggttcggttaatggtcacgagtttgaaatcgaaggcgagggagagggtcgtccatatgagggcactcagaccgctaaactcaaagtaacgaagggcggtccgcttccgttcgcctgggacattcttagcccacaatttatgtacggttctaaggcttacgtgaaacatcccgccgacattccagactatctgaaactgtcattccctgaaggtttcaaatgggaacgtgtgatgaacttcgaggatggcggtgtagtgaccgtcacccaagacagctcccttcaggacggcgaattcatttacaaagtaaagttgcgcggaaccaatttcccctccgatggcccagtaatgcaaaagaaaacgatgggatgggaagcatcctcggagcgtatgtatccggaggacggcgctttgaaaggagaaatcaaacagcgccttaagttgaaagacggtggccattacgacgcggaggtcaaaacgacctataaggctaagaaacctgttcagctgccgggtgcatataacgtcaacattaagttggacatcacttcccataacgaggattacaccatcgtggaacagtatgaacgtgcagagggccgccacagcactggcggcatggatgagctttacaagtaa

SEQ ID NO: 6 - protein, 236 aa, Unknown
  Discosoma sea anemone
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 7 - DNA, 33 bp, Escherichia coli
gccgctaatgacgaaaactatgcgcttgcagca

SEQ ID NO: 8 - protein, 11 aa, Escherichia coli
AANDENYALAA

SEQ ID NO: 9 - DNA, 33 bp, Artificial Sequence
  Synthethic polynucleotide
gcggcgaatgatgagaattacgctgacgcttcg

SEQ ID NO: 10 - protein, 11 aa, Artificial Sequence
  Synthetic polypeptide
AANDENYADAS

SEQ ID NO: 11 - DNA, 45 bp, Artificial Sequence
  Synthetic polynucleotide
gcggcgaatgatgagaattacagcgagaactacgctgacgcttcg

SEQ ID NO: 12 - protein, 15 aa, Artificial Sequence
  Synthetic polypeptide
AANDENYSENYADAS

SEQ ID NO: 13 - DNA, 45 bp, Artificial Sequence
  Synthetic polynucleotide
gcggcgaatgatgagaattacagcgagaactacgctgatcaaccg

SEQ ID NO: 14 - protein, 15 aa, Artificial Sequence
  Synthetic polypeptide
AANDENYSENYADQP

SEQ ID NO: 15 - DNA, 45 bp, Artificial Sequence
  Synthetic polynucleotide
gcggcgaatgatgagaattacagcgagaactacgctaagccatcg

SEQ ID NO: 16 - protein, 15 aa, Artificial Sequence
  Synthetic polypeptide
AANDENYSENYAKPS

SEQ ID NO: 17 - DNA, 45 bp, Artificial Sequence
  Synthetic polynucleotide
gcggcgaatgatgagaattacagcgagaactacgctgatggcgcg

SEQ ID NO: 18 - protein, 15 aa, Artificial Sequence
  Synthetic polypeptide
AANDENYSENYADGA

SEQ ID NO: 19 - DNA, 45 bp, Artificial Sequence
  Synthetic polynucleotide
gcggcgaatgatgagaattacagcgagaactacgctgatggctcg

SEQ ID NO: 20 - protein, 15 aa, Artificial Sequence
  Synthetic polypeptide
AANDENYSENYADGS

SEQ ID NO: 21 - DNA, 33 bp, Staphylococcus aureus
ggcaaatcaaacaataatttcgcagtagctgcc

SEQ ID NO: 22 - protein, 11 aa, Staphylococcus aureus
GKSNNNFAVAA

SEQ ID NO: 23 - DNA, 33 bp, Artificial Sequence
  Synthetic polynucleotide
ggcaaatcaaacaataatttcgcagacgcttcg

SEQ ID NO: 24 - protein, 11 aa, Artificial Sequence
  Synthetic polypeptide
GKSNNNFADAS

SEQ ID NO: 25 - DNA, 45 bp, Artificial Sequence
  Synthetic polynucleotide
ggcaaatcaaacaataatttcagcaacaatttcgcagacgcttcg

SEQ ID NO: 26 - protein, 15 aa, Artificial Sequence
  Synthetic polypeptide
GKSNNNFSNNFADAS

SEQ ID NO: 27 - DNA, 45 bp, Artificial Sequence
  Synthetic polynucleotide
ggcaaatcaaacaataatttcagcaacaatttcgcagatcaaccg

SEQ ID NO: 28 - protein, 15 aa, Artificial Sequence
  Synthetic polypeptide
GKSNNNFSNNFADQP

SEQ ID NO: 29 - DNA, 45 bp, Artificial Sequence
  Synthetic polynucleotide
ggcaaatcaaacaataatttcagcaacaatttcgcaaagccatcg

SEQ ID NO: 30 - protein, 15 aa, Artificial Sequence
  Synthetic polypeptide
GKSNNNFSNNFAKPS

SEQ ID NO: 31 - DNA, 45 bp, Artificial Sequence
  Synthetic polynucleotide
ggcaaatcaaacaataatttcagcaacaatttcgcagatggcgcg

SEQ ID NO: 32 - protein, 15 aa, Artificial Sequence
  Synthetic polypeptide
GKSNNNFSNNFADGA

SEQ ID NO: 33 - DNA, 45 bp, Artificial Sequence
  Synthetic polynucleotide
ggcaaatcaaacaataatttcagcaacaatttcgcagatggctcg

SEQ ID NO: 34 - protein, 15 aa, Artificial Sequence
  Synthetic polypeptide
GKSNNNFSNNFADGS

SEQ ID NO: 35 - protein, 15 aa, Artificial Sequence
  Synthetic polypeptide
AANDENYSENYADQA

SEQ ID NO: 36 - protein, 15 aa, Artificial Sequence
  Synthetic polypeptide
AANDENYSENYAQPS

SEQ ID NO: 37 - protein, 15 aa, Artificial Sequence
  Synthetic polypeptide
AANDENYSENYAHPP

SEQ ID NO: 38 - protein, 15 aa, Artificial Sequence
  Synthetic polypeptide
AANDENYSENYADQS

SEQ ID NO: 39 - protein, 15 aa, Artificial Sequence
  Synthetic polypeptide
AANDENYSENYAKNP

SEQ ID NO: 40 - protein, 15 aa, Artificial Sequence
  Synthetic polypeptide
AANDENYSENYAMKP

SEQ ID NO: 41 - protein, 11 aa, Artificial Sequence
  Synthetic polypeptide
AANDENYADQA

SEQ ID NO: 42 - protein, 11 aa, Artificial Sequence
  Synthetic polypeptide
AANDENYAQPS

SEQ ID NO: 43 - protein, 11 aa, Artificial Sequence
  Synthetic polypeptide
AANDENYAHPP

SEQ ID NO: 44 - protein, 11 aa, Artificial Sequence
  Synthetic polypeptide
AANDENYADQS

SEQ ID NO: 45 - protein, 11 aa, Artificial Sequence
  Synthetic polypeptide
AANDENYAKNP

SEQ ID NO: 46 - protein, 11 aa, Artificial Sequence
  Synthetic polypeptide
AANDENYAMKP

SEQ ID NO: 47 - protein, 15 aa, Artificial Sequence
  Synthetic polypeptide
GKSNNNFSNNFADQA

SEQ ID NO: 48 - protein, 15 aa, Artificial Sequence
  Synthetic polypeptide
GKSNNNFSNNFAQPS

SEQ ID NO: 49 - protein, 15 aa, Artificial Sequence
  Synthetic polypeptide
GKSNNNFSNNFAHPP

SEQ ID NO: 50 - protein, 15 aa, Artificial Sequence
  Synthetic polypeptide
GKSNNNFSNNFADQS

SEQ ID NO: 51 - protein, 15 aa, Artificial Sequence
  Synthetic polypeptide
GKSNNNFSNNFAKNP

SEQ ID NO: 52 - protein, 15 aa, Artificial Sequence
  Synthetic polypeptide
GKSNNNFSNNFAMKP

SEQ ID NO: 53 - protein, 11 aa, Artificial Sequence
  Synthetic polypeptide
GKSNNNFADQA

SEQ ID NO: 54 - protein, 11 aa, Artificial Sequence
  Synthetic polypeptide
GKSNNNFAQPS

SEQ ID NO: 55 - protein, 11 aa, Artificial Sequence
  Synthetic polypeptide
GKSNNNFAHPP

SEQ ID NO: 56 - protein, 11 aa, Artificial Sequence
  Synthetic polypeptide
GKSNNNFADQS

SEQ ID NO: 57 - protein, 11 aa, Artificial Sequence
  Synthetic polypeptide
GKSNNNFAKNP

SEQ ID NO: 58 - protein, 11 aa, Artificial Sequence
  Synthetic polypeptide
GKSNNNFAMKP